(19) United States
(12) Patent Application Publication
(10) Pub. No.: US 2026/0074071 A1
(43) Pub. Date: Mar. 12, 2026

(54) METHOD FOR ASSISTING A NEUROSURGICAL OPERATION AND ARRANGEMENT FOR ASSISTING A NEUROSURGICAL OPERATION

(71) Applicant: Carl Zeiss Meditec AG, Jena (DE)

(72) Inventors: Christian Dorfer, Vienna (AT); Stefan Saur, Aalen (DE)

(21) Appl. No.: 19/265,037

(22) Filed: Jul. 10, 2025

(30) Foreign Application Priority Data
Jul. 12, 2024 (EP) 24 188 373.5

(51) Int. Cl. G16H 50/20

(52) U.S. Cl. CPC G16H 50/20

(57) ABSTRACT

A method for assisting a neurosurgical operation, wherein at least one image representation of an operating region on a patient is captured by means of a medical visualization system wherein categorical position information describing an anatomical position of a tumor present in the operating region is acquired and/or obtained, wherein the at least one captured image representation and the acquired and/or obtained categorical position information are supplied to a trained machine learning model as input data, wherein a tumor type is predicted by means of the trained machine learning model using the at least one captured image representation and the acquired and/or obtained categorical position information as a starting point, wherein the trained machine learning model provides tumor type information describing the tumor type as output data, and wherein the tumor type information is output. The invention further relates to an arrangement for assisting a neurosurgical operation.

### CROSS-REFERENCE TO RELATED APPLICATIONS

The present application claims priority under 35 U.S.C. § 119 to European Patent Application No. 24 188 373.5, filed Jul. 12, 2024, the contents of which are incorporated by reference herein in their entirety.

The invention relates to a method for assisting a neurosurgical operation and to an arrangement for assisting a neurosurgical operation.

A suitable microsurgical strategy (degree of resection) must be chosen depending on the type of tumour. For example, glioblastomas are removed very aggressively, whereas the edges of tumours may be left in the case of metastases, as these can be treated post-surgery with radiotherapy or medication. Lymphomas are not even resected.

Even modern preoperative imaging techniques (MRI, CT, etc.) do not allow differentiation between tumour types, and so a surgeon starts the operation with a shortlist of potential tumour types (e.g. glioblastoma and metastasis). In neurosurgery, the type of tumour is only finally determined by a biopsy, within the scope of which tissue is taken and neuropathologically examined after the operation (post-operatively). For a first result still during surgery, a histopathological frozen section examination is now performed, which means 20 to 30 minutes waiting time during the procedure. This leads to an increase in the cost of the operation and to an increase in the risk to the patient as a result of prolonged anaesthesia. In addition, the significance of the histopathological frozen section examination is limited with an accuracy of ˜90%. In an alternative to the biopsy or in addition, measuring systems based on e.g. Raman spectroscopy or confocal endomicroscopy might also be used in the operating theatre. However, this requires additional hardware, which firstly generates investment costs and secondly also requires space in the operating theatre, which is usually limited.

WO 2023/156406 A1 describes a system comprising one or more processors and one or more memory apparatuses for training a machine learning algorithm, the system being configured to receive training data and the training data comprising images that show tissue; to adapt the machine learning algorithm on the basis of the training data such that the machine learning algorithm creates instruction data for at least a portion of the tissue shown in the images, the instruction data indicating an action to be performed on the tissue; and to provide the trained machine learning algorithm. Furthermore, a system for applying such a machine learning algorithm and corresponding procedures are described.

US 2016/0 035 093 A1 describes a multimodal brain mapping system (MBMS) comprising one or more scopes (e.g. microscopes or endoscopes) that are connected to one or more processors, wherein the one or more processors obtain training data from one or more first images and/or first data, with one or more abnormal regions and one or more normal regions being identified; furthermore receive a second image that was recorded by one or more of the endoscopes at a time later than the one or more first images and/or first data and/or recorded using a different imaging technique; and, using machine learning trained using training data, generate one or more visible indicators that identify one or more abnormalities in the second image, with the one or more visible indicators being generated in real time while the second image is created. One or more scopes display one or more visible indicators on the second image.

A classification of brain tumour types on the basis of MRI and microscope images is known from Efecan Cekic et al., Deep Learning-Assisted Segmentation and Classification of Brain Tumor Types on Magnetic Resonance and Surgical Microscope Images, World Neurosurgery, E1-E9, 2023.

Quinn T. Ostrom et al., CBTRUS Statistical Report: Primary Brain and Other Central Nervous System Tumors Diagnosed in the United States in 2016-2020, Neuro-Oncology, Volume 25, Supplementary Issue 4, October 2023, Pages iv1-iv99, https: //doi.org/10.1093/neuonc/noad149 provide statistical information on the frequency of tumour types in neurosurgery.

The problem addressed by the invention is that of proposing a solution in which a tumour type can be determined robustly and reliably during neurosurgical intervention, in particular in low-cost and cost-effective fashion.

According to the invention, the problem is solved by a method having the features of Claim 1 and an arrangement having the features of Claim 15. Advantageous configurations of the invention are evident from the dependent claims.

It is one of the basic ideas of the invention to use a trained machine learning model to predict a tumour type on the basis of at least one image representation captured by a medical visualization system. A further basic idea of the invention is that of taking a piece of categorical position information into account in the process by virtue of the categorical position information being acquired and/or obtained and supplied to the trained machine learning model, likewise as input data. The categorical position information describes an anatomical position of a tumour present in the operating region. It has been shown that the (categorical) anatomical position strongly correlates with a tumour type, and so a prediction accuracy can be significantly improved by taking the categorical position information into account. The trained machine learning model provides tumour type information that describes the tumour type as output data. The tumour type information is output. Provision can be made for the tumour type information to be displayed to the surgeon and/or an assistant on a display device. The surgeon can then choose the appropriate microsurgical strategy (degree of resection) on the basis of the tumour type information that is output and displayed in particular.

The method and the arrangement make it possible in particular to perform a prediction (classification) of the tumour type and a display of the result during the operation. The classification and/or display may be performed in particular while the surgeon and/or an assistant is inspecting the tissue, especially in real time or virtually in real time. Building on this, the surgeon may choose and/or change a resection strategy in particular. The prediction of the tumour type can therefore have a direct influence on the course of the operation.

In particular, a method for assisting a neurosurgical operation is made available, wherein at least one image representation of an operating region on a patient is captured by means of a medical visualization system, wherein categorical position information describing an anatomical position of a tumour present in the operating region is acquired and/or obtained, wherein the at least one captured image representation and the acquired and/or obtained categorical position information are supplied to a trained machine learning model as input data, wherein a tumour type is predicted by means of the trained machine learning model using the at least one captured image representation and the acquired and/or obtained categorical position information as a starting point, wherein the trained machine learning model provides tumour type information describing the tumour type as output data, and wherein the tumour type information is output.

Furthermore, an arrangement for assisting a neurosurgical operation, in particular, is developed, comprising a data processing device, wherein the data processing device is configured to obtain at least one image representation of an operating region on the patient, captured by means of a medical visualization system, to obtain categorical position information describing an anatomical position of a tumour present in the operating region, to provide a trained machine learning model, to supply the at least one captured image representation and the obtained categorical position information to the trained machine learning model as input data and to predict a tumour type by means of the trained machine learning model using the at least one captured image representation and the obtained categorical position information as a starting point, wherein the trained machine learning model provides tumour type information describing the tumour type as output data, and to output the tumour type information.

In addition to eliminating the disadvantages of the prior art, a further advantage of the method and the arrangement is that, in particular, the use of the categorical position information means that preoperative image data need not be evaluated (using a machine learning model), and hence a leaner configuration (especially of the machine learning model) in view of required resources (computing power and memory) is sufficient. Furthermore, the categorical position information is also in particular not reliably visible on/in the preoperative image data. From the preoperative image data, a location in the field of view (there) can be determined well. However, this does not necessarily allow an assignment to an anatomical position because it may not enable referencing to overarching anatomical structures under certain circumstances. In addition, the categorical position information can be easily determined by the physician themselves, or it is already available in the clinical workflow in any case. Since the evaluation of pre-operative images by means of machine learning methods is always accompanied by uncertainties, errors can moreover be avoided as a result of omitting the evaluation. The at least one piece of categorical position information furthermore implicitly brings about a normalization with respect to the anatomy. For instance, a predetermined voxel in an MRI image (e.g. voxel 10|10|10) may show different anatomical regions in different patients, depending on for example a patient's position and/or a region of coverage.

Categorical data, in contrast to metric data, do not have interval-scaled numerical values that allow computational operations. Categorical features are described by nominal and ordinal scales, in particular by at least one nominal scale in the present case. In particular, a piece of categorical position information contains an anatomical position on a nominal scale. The nominal scale denotes or relates to (anatomical) regions in the human body in particular. For the purpose of simpler processing by the machine learning model, the (anatomical) regions can be expressed or encoded as numerical values in particular, wherein the properties of the nominal scale remain.

A medical visualization system is a surgical microscope in particular, especially a microsurgical surgical microscope, or a confocal laser microscope. The surgical microscope may be a digital surgical microscope, for example, in particular with a digital visualization chain, in which there is imaging on (at least) one camera sensor. In addition, the surgical microscope may be an analogue surgical microscope, which in particular has an optical path with lenses and eyepiece(s). Furthermore, the surgical microscope may be a hybrid surgical microscope. However, in principle, a medical visualization system may also be a different medical system that is capable of capturing at least one image representation of the operating region, for instance a micro-inspection tool or an endoscope. The medical visualization system is used on living tissue in particular, i.e. the examination is carried out in vivo in particular.

In particular, the method is carried out on living tissue, i.e. the method is carried out in vivo in particular. In particular, the arrangement or the medical visualization system is used on living tissue, i.e. the arrangement or the medical visualization system is used in vivo in particular.

The at least one image representation may be captured and/or provided by the medical visualization system. The at least one captured image may comprise a white light image and/or a fluorescence image, for example BLUE-400 or YELLOW-560. For example, provision can be made for a white light image and a fluorescence image to be fused together and used as a (fused) captured image representation. Expediently, the captured image representation is supplied to the trained machine learning model.

For example, the machine learning model may be an artificial neural network or at least comprise one. The captured at least one image representation and the categorical position information are supplied to the machine learning model as input data. The machine learning model provides a prediction of the tumour type as output data. The machine learning model is or has been trained with the aid of training data in particular. In this case, the training data in particular comprise a multiplicity of pairs, each of which comprises an image representation showing the tumour and a piece of categorical position information describing the anatomical position of the tumour depicted, coupled with tumour type information as annotation or as ground truth. The ground truth can be determined by means of a neuropathological tissue examination, for example. The machine learning model is trained with the aid of the training data in a manner known per se, in particular by way of supervised learning.

Provision can be made for the machine learning model to be pretrained with the aid of statistical metadata relating to the correlation between anatomical position (in the form of categorical position information) and tumour type. This allows the machine learning model to learn the prevalence statistics of the tumour types depending on the anatomical position. Subsequently, within fine tuning, the training can be implemented using the training data, comprising the image representations, the categorical position information and the tumour type assigned as ground truth in each case. For example, statistical reports such as the aforementioned report (Ostrom et al.) may be used as a data source for the prevalences.

For example, the tumour type information may comprise probability values for possible tumour types, as estimated by the trained machine learning model for each tumour type. The probability values can then be used to determine the most likely tumour type present. However, provision may also be made for the tumour type information to already contain a statement about the tumour type present without probability values being contained in the tumour type information (only the most probable tumour type is specified in that case).

Provision can be made for the method to only be started after a user (surgeon or assistant) has given the order in this respect. For example, this may be implemented once tissue located above the tumour has been removed or opened up and the tumour has been reached or once the tumour can be visually detected.

Parts of the arrangement, in particular the data processing device, may be designed, either individually or together, as a combination of hardware and software, for example as program code that is executed on a microcontroller or microprocessor. However, parts may also be designed, either individually or together, as an application-specific integrated circuit (ASIC) and/or field-programmable gate array (FPGA) and/or graphics processor unit (GPU) and/or digital signal processor (DSP). In particular, the data processing device comprises at least one computing device and at least one memory.

An embodiment provides for the categorical position information to be acquired and/or obtained in text-based fashion. This allows the anatomical position information to be acquired and/or obtained in a manner that is particularly easily understood by a human. For example, provision can be made for the categorical position information to be acquired in text-based fashion on a display and operating device (GUI), wherein a user (in particular a member of the clinical team, for example a surgeon or an assistant) may enter the categorical position information on a keyboard. Furthermore, the categorical position information may be selected from a selection list in which, for example, a plurality of categories for the categorical position information are specified in text-based fashion. Furthermore, the categorical position information may be acquired as transcribed voice input.

An embodiment provides for an anatomical map to be displayed for the purpose of acquiring the categorical position information, wherein the categorical position information is acquired by selection on the anatomical map. This may provide a particularly descriptive type of acquisition, whereby in particular an acquisition speed may be increased, and incorrect inputs may be prevented or at least reduced. For example, provision can be made for the anatomical map to be displayed on a display device or a display and operating device. A user (surgeon or assistant) may specify the categorical position information by selecting the anatomical position on the anatomical map.

An embodiment provides for the categorical position information to comprise information regarding the brain lobe containing the tumour. This can increase the quality of the prediction. In particular, the information regarding the brain lobe comprises one of the following specifications: frontal lobe (lobus frontalis), temporal lobe (lobus temporalis), parietal lobe (lobus parietalis), occipital lobe (lobus occipitalis).

An embodiment provides for the categorical position information to comprise information regarding the half of the brain containing the tumour. This can increase the quality of the prediction. In particular, the information regarding the half of the brain comprises one of the following specifications: left hemisphere, right hemisphere.

An embodiment provides for the categorical position information to comprise information regarding the brain region containing the tumour. This can increase the quality of the prediction. In particular, the information regarding the brain region comprises one of the following specifications: supratentorial, supratentorial midline, infratentorial, periventricular, spinal (i.e. in the border region between the brain and spine), central.

Further, provision can be made for the information to comprise one of the following pieces of information for a location within the brain lobe, half of the brain and/or brain region, in addition to the above or in an alternative: anterior vs. posterior; medial vs. lateral, cortical/subcortical (superficial) or deep; midline or not.

An embodiment provides for the categorical position information to comprise at least one piece of relational information that locates the tumour in relation to anatomical features. This allows a categorical specification to be made even more accurate. This may further increase the quality of the prediction. For example, provision can be made for the relational information to contain specifications regarding a position relative to a vascular system of the brain, for example a specification that the tumour is located between two vessels (with corresponding anatomical specification) or that the tumour is in contact with a vessel (with anatomical specification), etc. Provision can be made for the relational information to be given with reference to a digital twin of (at least) one part of the patient or a generic digital twin of (at least) one part of the human body.

An embodiment additionally provides for at least one visual indicator to be generated and output as an image signal, wherein the visual indicator identifies at least one region in the captured at least one image representation that was taken into account in the prediction process. For example, the visual indicator can be created or determined on the basis of data from the trained machine learning model. For example, what are known as attention maps (cf. e.g. Jungkan An et al., Attention Map-Guided Visual Explanations for Deep Neural Networks, Appl. Sci. 2022, 12(8), 3846; https://doi.org/10.3390/app12083846) and/or class activation maps (cf. Anh Pham Thi Minh, Overview of Class Activation Maps for Visualization Explainability, arXiv:2309.14304 [cs.CV], 2023, https://doi.org/10.48550/arXiv.2309.14304) may be used to this end. Provision can be made for the visual indicator to be displayed together with the at least one image representation, for example by virtue of the visual indicator being displayed over the respective region of the at least one image representation. An embodiment provides for at least one piece of patient information to be acquired and/or obtained, wherein the trained machine learning model is additionally supplied with the at least one acquired and/or obtained piece of patient information as input data, and wherein the trained machine learning model predicts the tumour type additionally taking into account the at least one piece of patient information. This may further improve the quality of the prediction, as the occurrence of tumour types in particular also correlates with the characteristics of the patients. For example, a piece of patient information may concern one of the following: age, gender, a specification as to whether a contrast agent has accumulated and/or a history of the disease (e.g. occurrence of epilepsy or whether another primary tumour, for example at the same location, has already been diagnosed in the patient, etc.). When training the machine learning model, the training data used in the process in particular also comprise the corresponding at least one piece of patient information.

Provision can be made for the machine learning model to be pretrained by means of statistical metadata that comprise at least one piece of patient information paired with the respective frequencies of the tumour types, as has already been described above.

Provision can be made for preoperative data (e.g. MRI, CT, etc.) of the operating region to be also taken into account. For example, the preoperative data may be taken into account as patient information. The preoperative data are also supplied to the trained machine learning model as input data and taken into account in the prediction of the tumour type. The training data comprise corresponding preoperative data in that case.

An embodiment provides for the trained machine learning model to comprise a convolutional neural network, a multi-layer perceptron and a classification network, wherein the acquired and/or obtained at least one image representation is converted into a first embedding part by means of the convolutional neural network, wherein the categorical position information and/or the at least one piece of patient information is converted into a second embedding part by means of the multi-layer perceptron, and wherein the first embedding part and the second embedding part are supplied to the classification network as input data. This allows a particularly high quality of prediction to be achieved.

In principle, however, the machine learning model may also have a different architecture, for example a transformer architecture. Moreover, approaches from the field of multi-sensor fusion may also be used (cf. Benedict Marsh et al., A Critical Review of Deep Learning-Based Multi-Sensor Fusion Techniques, Sensors 2022, 22(23), 9364; https://doi.org/10.3390/s22239364).

An embodiment provides for sound data relating to an aspirator used during the neurosurgical operation to be acquired and/or obtained, wherein the trained machine learning model is additionally supplied with the acquired and/or obtained sound data as input data, and wherein the trained machine learning model predicts the tumour type additionally taking into account the acquired and/or obtained sound data. This may further increase the quality of the prediction since the sound information also correlates with the tumour type on account of different tissue types. The sound data comprise in particular recorded sound signals, which are acquired by means of a microphone, for example. When training the machine learning model, the training data used in the process in particular also comprise corresponding sound data.

An embodiment provides for haptic measurement data representing an elasticity of a tissue to be acquired and/or obtained in the operating region, wherein the trained machine learning model is additionally supplied with the acquired and/or obtained haptic measurement data as input data, and wherein the trained machine learning model predicts the tumour type additionally taking into account the acquired and/or obtained haptic measurement data. This may further increase the quality when predicting the tumour type. When training the machine learning model, the training data used in the process in particular also comprise corresponding haptic measurement data. The haptic measurement data can be acquired by means of piezoelectric sensors, for example (cf. e.g. Jörg Wallaschek et al., Mechatronischer Tastsinn—Wie piezoelektrische taktile Sensoren Diagnosen optimieren, AlumniCampus 4, 2010, https://www.uni-hannover.de/fileadmin/luh/content/alumni/alumnicampus/AC_4_2010/48-51_forsch10_wallaschek. pdf).

An embodiment provides for a list of tumour types to be acquired and/or predetermined, wherein the trained machine learning model is additionally supplied with the list of tumour types as input data, and wherein the trained machine learning model predicts the tumour type additionally taking into account the list of tumour types. This may increase a quality of the prediction because the trained machine learning model receives additional information regarding the tumour types that may be present, i.e. it has to make a decision only between a limited number of tumour types or has to estimate probabilities only for this limited number of tumour types. When training the machine learning model, the training data used in the process in particular also comprise corresponding lists.

An embodiment provides for a list of tumour types to be acquired and/or predetermined, wherein at least groupwise different trained machine learning models are used for different lists of tumour types, wherein the respective appropriate trained machine learning model is selected on the basis of the acquired and/or predetermined list of tumour types. This may further increase a quality of the prediction because a specialized machine learning model can be provided for different lists. The respective appropriate or specialized machine learning model then assesses the tumour type, especially only for the tumour types contained in the associated list. However, provision may also be made for shared machine learning models to be used for lists that overlap one another with respect to tumour types, i.e., in principle, a plurality of lists may share a common machine learning model.

A refined embodiment provides for the list to comprise two tumour types. This allows the highest degree of specialization of the trained machine learning models to be implemented, and so a quality of the prediction can also be further increased. In particular, provision can be made for a trained machine learning model to be provided and used for each combination of two tumour types.

An embodiment provides for at least one control signal to be created and/or provided using the predicted tumour type as a starting point, said control signal being designed to control the medical visualization system and/or another medical apparatus. This allows a surgeon to be assisted more comprehensively in the upcoming phase of the operation (in particular tumour resection). For example, the at least one control signal may serve to change settings of the medical visualization system and/or activate and/or prepare another medical apparatus.

Provision can be made for the machine learning model to be retrained with the aid of biopsy data containing the respective (laboratory-)determined tumour type. The biopsy data then serve as ground truth for the respective correspondingly captured image representation.

Further features relating to the configuration of the arrangement arise from the description of configurations of the method. Here, the advantages of the arrangement are in each case the same as in the configurations of the method.

The invention is explained in detail below on the basis of preferred exemplary embodiments with reference to the figures. In the figures:

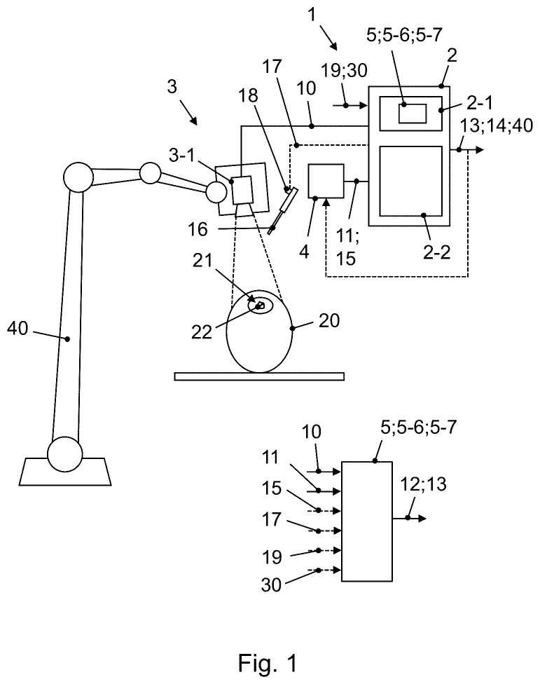

FIG. 1 shows a schematic illustration of an embodiment of the arrangement for assisting a neurosurgical operation;

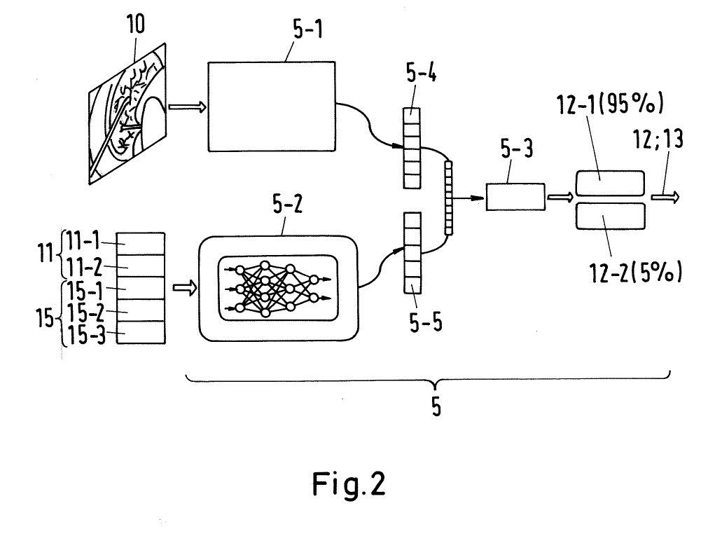

FIG. 2 shows an exemplary embodiment of the machine learning model;

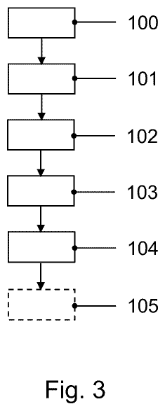

FIG. 3 shows a schematic flowchart of an embodiment of the method.

FIG. 1 shows a schematic illustration of an embodiment of the arrangement 1 for assisting a neurosurgical operation. The arrangement 1 is configured to carry out the method described in this disclosure. The method is described in detail below on the basis of the arrangement 1.

The arrangement 1 comprises a data processing device 2. The data processing device 2 comprises at least one computing device 2-1 and at least one memory 2-2. The computing device 2-1 is configured to carry out computing operations required for performing the method and may for this purpose access data stored in the memory 2-2.

The data processing device 2 is configured to obtain at least one image representation 10 of an operating region 21 on the patient 20 captured by means of a medical visualization system 3, for example a surgical microscope, in particular by means of a camera 3-1. The medical visualization system 3 may also be part of the arrangement 1. Furthermore, the medical visualization system 3 may also comprise the arrangement 1.

Furthermore, the data processing device 2 obtains categorical position information 11 describing an anatomical position of a tumour 22 present in the operating region 21. For example, the categorical position information 11 may be acquired by means of a display and operating device 4. The display and operating device 4 may be part of the arrangement 1. Alternatively, the categorical position information 11 may also be provided by a hospital information system (HIS) and/or retrieved from the latter by means of the data processing device 2. In particular, the categorical position information 11 may be provided by and/or retrieved from an electronic health record (EHR).

The data processing device 2 is configured to provide a trained machine learning model 5, supply the at least one captured image representation 10 and the obtained categorical position information 11 to the trained machine learning model 5 as input data and predict a tumour type 12 by means of the trained machine learning model 5 on the basis of the at least one captured image representation 10 and the obtained categorical position information 11. At an output, the trained machine learning model 5 provides tumour type information 13 that describes the tumour type 12 as output data. The data processing device 2 outputs the tumour type information 13. For example, the data processing device 2 may output the tumour type information 13 on a display unit, e.g. on the display and operating device 4 or on any (other) display. The display unit may be a display unit of a surgical microscope or an external 2D or 3D monitor. Furthermore, there may also be an overlay in a digital eyepiece or in AR or VR glasses (head-mounted display, HMD).

In particular, provision is made for the tumour type information 13 to be displayed, for example on the display and operating device 4. For example, the tumour type information 13 may be displayed as text information that encompasses the tumour type. Provision can be made for probabilities associated with the presence of different tumour types 12 to be also output and/or displayed. On the basis of the displayed tumour type information, a surgeon is then able to decide how the operation should be continued, in particular which degree of resection is selected for the present tumour 22.

Provision can be made for the categorical position information 11 to be acquired and/or obtained in text-based fashion. For example, provision can be made for the categorical position information 11 to be acquired directly as text by means of a keyboard, for example by means of the display and operating device 4. In an alternative to that or in addition, provision can also be made for the categorical position information 11 to be selectable in text-based fashion from a list, for example by means of the display and operating device 4.

In an alternative to that or in addition, provision can be made for an anatomical map to be displayed for the purpose of acquiring the categorical position information 11, wherein the categorical position information 11 is acquired by selection on the anatomical map. For example, provision can be made for the anatomical map to be displayed on the display and operating device 4, wherein anatomical regions are selectable, for example by virtue of these being selected by clicking and/or touching a corresponding region on the display and operating device 4.

Provision can be made for the categorical position information 11 to comprise information regarding the brain lobe containing the tumour.

Provision can be made for the categorical position information 11 to comprise information regarding the half of the brain containing the tumour.

Provision can be made for the categorical position information 11 to comprise information regarding the brain region containing the tumour.

Provision can be made for the categorical position information 11 to comprise at least one piece of relational information that locates the tumour in relation to anatomical features.

Provision can be made for at least one visual indicator 14 to be additionally generated and output as an image signal, wherein the visual indicator 14 identifies at least one region in the captured at least one image representation 10 that was taken into account in the prediction process. For example, provision can be made for the at least one visual indicator 14 to be displayed on the display and operating device 4, in particular together with the detected at least one image representation 10.

Provision can be made for at least one piece of patient information 15 to be acquired and/or obtained, wherein the trained machine learning model 5 is additionally supplied with the at least one acquired and/or obtained piece of patient information 15 as input data, wherein the trained machine learning model 15 predicts the tumour type 12 additionally taking into account the at least one piece of patient information 15. For example, the at least one piece of patient information 15 may be extracted from a digital health record. For example, provision can be made for the at least one piece of patient information 15 to be queried from a database.

FIG. 2 shows an exemplary embodiment of the machine learning model 5. This embodiment provides for the trained machine learning model 5 to comprise a convolutional neural network 5-1, a multi-layer perceptron 5-2 and a classification network 5-3, wherein the acquired and/or obtained at least one image representation 10 is converted into a first embedding part 5-4 by means of the convolutional neural network 5-1, wherein the categorical position information 11 and/or the at least one piece of patient information 15 is converted into a second embedding part 5-5 by means of the multi-layer perceptron 5-2, and wherein the first embedding part 5-4 and the second embedding part 5-5 are supplied to the classification network 5-3 as input data. The categorical position information 11 for example comprises a specification of the brain lobe 11-1 and a specification of the brain region 11-2 (side). The at least one piece of patient information 15 for example comprises a gender 15-1, an age 15-2 and a history 15-3 regarding the tumour. Provision can be made for the multi-layer perceptron 5-2 to be pretrained with the aid of statistical data regarding the prevalence or frequency of tumour types for the respective manifestations of the categorical position information 11 and/or the patient information 15 (see below). For example, the classification network 5-3 estimates a probability of the presence of a respective tumour type 12. In the example shown, a prediction should be made as to whether a glioblastoma 12-1 or a metastasis 12-2 is present. By way of example, the prediction of the classification network 5-3 should result in a glioblastoma 12-1 being present with a probability of 95% and a metastasis being present with a probability of 5%. These values may be output as tumour type information 13.

There are multiple options for pre-training the multi-layer perceptron 5-2, one of which is carried out by way of example: For example, Monte Carlo sampling may be used to create data sets that follow the prevalence statistics. This allows the lower branch to be trained in that case. For this purpose, a classifier is used directly after the second embedding part 5-5. The result is a parametrization of the multi-layer perceptron 5-2. For training with real patient data (i.e. the at least one image representation 10 and the categorical position information 11), either the weights of the multi-layer perceptron 5-2 are subsequently frozen (all or only part) and/or a change in the weights is limited.

Provision can be made for sound data 17 (FIG. 1) relating to an aspirator 16 used during the neurosurgical operation to be acquired and/or obtained, wherein the trained machine learning model 5 is additionally supplied with the acquired and/or obtained sound data 17 as input data, and wherein the trained machine learning model 5 predicts the tumour type 12 additionally taking into account the acquired and/or obtained sound data 17. For example, the sound data 17 may be acquired by means of a microphone 18 arranged in the vicinity of or on the aspirator 16.

Provision can be made for haptic measurement data 19 representing an elasticity of a tissue to be acquired and/or obtained in the operating region 21, wherein the trained machine learning model 5 is additionally supplied with the acquired and/or obtained haptic measurement data 19 as input data, wherein the trained machine learning model 5 predicts the tumour type 12 additionally taking into account the acquired and/or obtained haptic measurement data 12.

Provision can be made for a list 30 of tumour types 12 to be acquired and/or predetermined, wherein the trained machine learning model 5 is additionally supplied with the list 30 of tumour types 12 as input data, and wherein the trained machine learning model 5 predicts the tumour type 12 additionally taking into account the list 30 of tumour types 12. In particular, the list 30 of tumour types 12 comprises a plurality of possible tumour types 12. For example, the list 30 may be transferred to the machine learning model 5 as a column vector, in which each entry corresponds to a possible tumour type 12 and the entries of the list 30 are selected by way of entering a “0” (=“This tumour type is not possible”) or a “1” (=“This tumour type is possible”). For example, the column vector (0, 1, 0, 0, 1) for tumour types 1 to 5 specifies that only tumour type 2 and tumour type 5 can be present, and therefore the trained machine learning model 5 only has to make a prediction to distinguish between these two tumour types. The machine learning model 5 is trained during a training phase, in particular taking into account the corresponding lists 30.

Provision can be made for a list 30 of tumour types 12 to be acquired and/or predetermined, wherein at least groupwise different trained machine learning models 5 are used for different lists 30 of tumour types 12, wherein the respective appropriate trained machine learning model 5 is selected on the basis of the acquired and/or predetermined list 30 of tumour types 12. If in a much simplified example there are tumour types 1 to 4, which however only occur in the combinations of tumour type 1 with tumour type 2 and tumour type 3 with tumour type 4, then two specialized machine learning models 5 are used, wherein a first machine learning model 5-6 is specialized in predicting the first combination and a second machine learning model 5-7 is specialized in predicting the second combination. Accordingly, the list 30 specifies which of the combinations is present, e.g. in the form of a column vector as (1, 1, 0, 0) for the first combination or (0, 0, 1, 1) for the second combination. The trained first machine learning model 5-6 is used to predict the tumour type 12 in the first combination or in the first column vector; the trained second machine learning model 5-7 is used to predict the tumour type 12 in the second combination or in the second column vector.

In particular, provision is made for the list 30 to comprise two tumour types 12. This allows a maximum limitation of the possible tumour types to be undertaken. In particular, when a plurality of specialized machine learning models 5 are used, these may be maximally specialized on the respective combination present. Examples thereof include: glioblastoma vs. metastases, glioblastoma vs. lymphoma, lymphoma vs. astrocytoma and lymphoma vs. other tissue (the prediction therefore only includes a statement about whether or not a lymphoma is present).

The list 30 may for example be acquired on the display and operating device 4 or by means of another acquisition device (not shown). Provision can be made for a selection of lists 30 to be displayed on the display and operating device 4 and be able to be selected. However, provision can also be made for a user to be able to specify the list 30 by selecting possible tumour types 12.

Provision can be made for at least one control signal 40 to be created and/or provided using the predicted tumour type 12 as a starting point, said control signal being designed to control the medical visualization system 3 and/or another medical apparatus (not shown). To this end, the data processing device 2 may be configured to create and provide at least one control signal 40 from the predicted tumour type 12 and/or from the tumour type information 13. The control signal 40 may be analogue and/or digital. For example, provision can be made for an operating mode of the medical visualization system 3 and/or the other medical apparatus to be set and/or modified and/or settings to be changed by means of the control signal 40.

FIG. 3 shows a schematic flowchart of an embodiment of the method for assisting a neurosurgical operation. The method is performed for example by means of an arrangement according to an embodiment as shown in FIG. 1. Provision can be made for it to be necessary for a user to start the method, for example if the tumour is exposed from among other tissue over the course of the operation, and said tumour can be captured visually. Furthermore, provision can also be made for the method to be started automatically when the tumour is exposed, for example when it is automatically detected that a tumour is visible in the operating region. In particular, the method may be provided and/or started within the scope of a function of the medical visualization system for automatic classification of a tumour type.

In a method step 100, at least one image representation of an operating region on a patient is captured by means of a medical visualization system. This is implemented on living tissue (in vivo) in particular. The at least one image representation is in particular a white light image, which is captured by means of the medical visualization system. In particular, this can be implemented automatically once the method or the function of the medical visualization system has been started.

Categorical position information that describes an anatomical position of a tumour present in the operating region is acquired and/or obtained in a method step 101.

Provision can also be made for the method step 101 to be carried out before the method step 100. In other words: The categorical position information may also be acquired and/or obtained before the start of the operation. Furthermore, the categorical position information may also be acquired and/or obtained after the start of the operation but before the acquisition of at least one image representation.

In addition, the method steps 100 and 101 may also be carried out in parallel or simultaneously.

In a method step 102, the at least one captured image representation and the acquired and/or obtained categorical position information are supplied to a trained machine learning model as input data.

In a method step 103, a tumour type is predicted by means of the trained machine learning model using the at least one captured image representation and the acquired and/or obtained categorical position information as a starting point, wherein the trained machine learning model provides tumour type information describing the tumour type as output data.

The tumour type information is output in a method step 104. In particular, provision can be made for the tumour type information to be output on the medical visualization system. For example, provision can be made for this to be output on a display device of the medical visualization system and/or in the form of an augmented image representation. The augmented image representation may in particular comprise a captured image representation (in particular a live image) of the operating region.

The method steps 101, 102, 103 and 104 may be implemented in real time or virtually in real time (<1 s), and so the surgeon and/or assistants can immediately, i.e. without a great delay, receive a result (tumour type information) and for example select and/or modify a resection strategy on the basis thereof. In particular, the result (the tumour type information) may be provided, in particular output and/or displayed, while the surgeon and/or the assistants examine the tissue (in vivo). This can improve the workflow of the operation.

In a method step 105, provision can be made for at least one control signal to be created and/or provided using the predicted tumour type as a starting point, said control signal being designed to control the medical visualization system and/or another medical apparatus.

Alternatively, provision can also be made for the method to be performed continuously and/or repeatedly (e.g. in the background), wherein the tumour type information is output upon reaching a predetermined threshold value of the classification accuracy, for example ˜90%.

Further embodiments of the method have already been described with reference to the arrangement.

### List of Reference Signs

1 Arrangement
2 Data processing device
2-1 Computing device
2-2 Memory
3 Medical visualization system
3-1 Camera
4 Display and operating device
5 Trained machine learning model
5-1 Convolutional neural network
5-2 Perceptron
5-3 Classification network
5-4 First embedding part
5-5 Second embedding part
5-6 Trained first machine learning model
5-7 Trained second machine learning model
10 Image representation
11 Categorical position information
11-1 Specification regarding the brain lobe
11-2 Specification regarding the brain region
12 Tumour type
12-1 Glioblastoma
12-2 Metastasis
13 Tumour type information
14 Visual indicator
15 Patient information
15-1 Gender
15-2 Age
15-3 History
16 Aspirator
17 Sound data
18 Microphone
19 Haptic measurement data
20 Patient
21 Operating region
22 Tumour
30 List (of tumour types)
40 Control signal
100-105 Method steps

1. A method for assisting a neurosurgical operation, the method comprising:
capturing at least one image representation of an operating region on a patient by a medical visualization system,
acquiring and/or obtaining categorical position information describing an anatomical position of a tumor present in the operating region 21) is acquired and/or obtained,
supplying the at least one captured image representation and the acquired and/or obtained categorical position information to a trained machine learning model as input data,
predicting a tumor type by the trained machine learning model using the at least one captured image representation and the acquired and/or obtained categorical position information as a starting point,
providing, by the trained machine learning model, tumor type information describing the tumor type as output data, and
outputting the tumor type information.

2. The method according to claim 1, wherein the categorical position information is acquired and/or obtained in text-based fashion.

3. The method according to claim 1, wherein an anatomical map is displayed for the purpose of acquiring the categorical position information, wherein the categorical position information is acquired by selection on the anatomical map.

4. The method according to claim 1, wherein the categorical position information comprises information regarding the brain lobe containing the tumor.

5. The method according to claim 1, wherein the categorical position information comprises information regarding the brain region containing the tumor.

6. The method according to claim 1, wherein the categorical position information comprises at least one piece of relational information that locates the tumor in relation to anatomical features.

7. The method according to claim 1, further comprising generating and outputting at least one visual indicator as an image signal, wherein the visual indicator identifies at least one region in the captured at least one image representation that was taken into account in the prediction process.

8. The method according to claim 1, wherein at least one piece of patient information is acquired and/or obtained, wherein the trained machine learning model is additionally supplied with the at least one acquired and/or obtained piece of patient information as input data, and wherein the trained machine learning model predicts the tumor type additionally taking into account the at least one piece of patient information.

9. The method according to claim 1, wherein the trained machine learning model comprises a convolutional neural network a multi-layer perceptron and a classification network, wherein the acquired and/or obtained at least one image representation is converted into a first embedding part by means of the convolutional neural network, wherein the categorical position information and/or the at least one piece of patient information is converted into a second embedding part by means of the multi-layer perceptron, and wherein the first embedding part and the second embedding part are supplied to the classification network as input data.

10. The method according to claim 1, further comprising acquiring and/or obtaining sound data relating to an aspirator used during the neurosurgical operation wherein the trained machine learning model is additionally supplied with the acquired and/or obtained sound data as input data, and wherein the trained machine learning model predicts the tumor type additionally taking into account the acquired and/or obtained sound data.

11. The method according to claim 1, further comprising acquiring and/or obtaining haptic measurement data representing an elasticity of a tissue in the operating region, wherein the trained machine learning model is additionally supplied with the acquired and/or obtained haptic measurement data as input data, and wherein the trained machine learning model predicts the tumor type additionally taking into account the acquired and/or obtained haptic measurement data.

12. The method according to claim 1, further comprising acquiring and/or predetermining a list of tumor types, wherein the trained machine learning model is additionally supplied with the list of tumor types as input data, and wherein the trained machine learning model predicts the tumor type additionally taking into account the list of tumor types.

13. The method according to claim 1, further comprising acquiring and/or predetermining a list of tumor types, wherein at least groupwise different trained machine learning models are used for different lists of tumor types, wherein the respective appropriate trained machine learning model is selected on the basis of the acquired and/or predetermined list of tumor types.

14. The method according to claim 1, wherein at least one control signal is created and/or provided using the predicted tumor type as a starting point, said control signal being designed to control the medical visualization system and/or another medical apparatus.

15. An arrangement for assisting a neurosurgical operation, comprising:
a data processing device, wherein the data processing device is configured to:
obtain at least one image representation of an operating region on the patient, captured by means of a medical visualization system,
obtain categorical position information describing an anatomical position of a tumor present in the operating region,
provide a trained machine learning model,
supply the at least one captured image representation and the obtained categorical position information to the trained machine learning model as input data,
predict a tumor type by means of the trained machine learning model using the at least one captured image representation and the obtained categorical position information as a starting point, wherein the trained machine learning model provides tumor type information describing the tumor type as output data, and
output the tumor type information.

16. The arrangement according to claim 15, wherein the arrangement comprises the medical visualization system.
